(19) United States
(12) Patent Application Publication
(10) Pub. No.: US 2010/0106255 A1
(43) Pub. Date: Apr. 29, 2010

(54) SELF-EXPANDING FRONTAL SINUS STENT AND INSERTION TOOL

(76) Inventor: Marc G. DUBIN, Pikesville, MD (US)

Correspondence Address: Pepper Hamilton LLP, 400 Berwyn Park, 899 Cassatt Road, Berwyn, PA, 19312-1183, US

(21) Appl. No.: 12/492,439

(22) Filed: Jun. 26, 2009

Related U.S. Application Data
(60) Provisional application No. 61/108,053, filed on Oct. 24, 2008.

(51) Int. Cl. A61F 2/04; A61M 5/00

(52) U.S. Cl. 623/23.7; 604/8

(57) ABSTRACT

A self-expanding stent for use in maintaining patency of the frontal sinus drainage pathway and for the management of the frontal sinus postoperatively. The self-expanding frontal sinus stent helps maintain the pathway connecting the frontal sinus cavity open by filling this space and preventing restenosis of the frontal sinus drainage pathway following sinus surgery. The self-expanding stent may include a medical grade, flexible plastic material having with one or more recoil mechanisms having a memory to self expand. The self-expanding stent is compliant meaning that it may expand to the actual size of the space into which it is placed. The self-expanding stent may also be self-retaining. An insertion tool having the appropriate angulation and malleability may be used for ease of insertion of the self-expanding stent endoscopically into the frontal sinus. The use of a self-expanding frontal sinus stent and insertion tool for ease of insertion leads to less trauma for the patient and better surgical outcomes.

### TECHNOLOGY FIELD

The present invention relates generally to a device for stenting of the frontal sinus, and more particularly to a self-expanding stent for use in keeping the frontal sinus pathway open and an insertion tool having the appropriate angulation and malleability to facilitate ease of insertion of the stent into the frontal sinus. This technology is particularly suited, but by no means limited, for use following frontal sinus surgery.

### BACKGROUND

Sinus surgeries are typically performed to remove tumors, to open up the sinus passageways, and to restore normal drainage of the sinuses. Sinus surgery may be performed endoscopically or externally. Endoscopic sinus surgery, for example, is a minimally invasive surgical procedure that may be performed to open up sinus air cells and sinus ostia (openings) with an endoscope. An external approach to sinus surgery is generally more invasive and the osteoplastic flap procedure is one widely accepted method of performing external frontal sinus surgery.

As stated above, one purpose of sinus surgery is to restore normal drainage of the sinuses. Normal function of the sinuses requires ventilation through the ostia (mouth-like opening) and is facilitated by a mucociliary transport process that maintains a constant flow of mucus out of the sinuses. All sinuses need ventilation to prevent infection and inflammation, a condition known as sinusitis. In healthy individuals, sinus ventilation occurs through the ostia into the nose. The sinuses open into the middle meatus (curved passage in each nasal cavity) under the middle turbinate (thin, bony process that is the lower portion of the ethmoid bone in each nasal cavity), which together are known as the osteomeatal complex, a key area of the nose. The hair-like cilia direct the flow of mucus toward the ostia.

Sinusitis develops when there is a problem in the area where the maxillary and frontal sinuses meet near the nose. When sinusitis occurs, the cilia work less efficiently, preventing the proper flow of mucus. The mucous membranes of the sinuses become engorged, resulting in ostia closure. Poor ventilation and accumulation of mucus then produce the conditions required for bacterial infection. During sinus surgery, a doctor opens the sinuses to alleviate problems with sinusitis.

Frontal sinus surgery is one of the most challenging areas of sinus surgery due to the difficult visualization and complex anatomy. The ultimate success or failure of frontal sinus surgical procedures, whether they be endonasal or external, is determined essentially by the rate of restenosis of the frontal sinus outflow tract or neo-ostium postoperatively. It is known that long-term stenting for a period of several months significantly reduces the rate of restenosis. However, rarely is stenting considered due to the lack of adequate commercially available products and lack of training.

There are currently no good commercially available options for short or long term stenting of the frontal sinus following sinus surgery. Also, because a circumferential injury is created in a narrow passage of the sinus, there is significant risk of scarring. This risk necessitates debridement in the post-operative period to keep the blood that forms in the area from turning into scar tissue that would necessitate additional surgery. This is uncomfortable but necessary for the patient. Frontal stenting is currently considered for failed attempts at keeping the frontal sinus pathway open.

One commercially available frontal sinus stent is the “Rains stent.” The Rains stent is described in U.S. Pat. No. 5,693,065 and includes a silicone rubber tube with an egg-shaped bulb on one end. The Rains stent may be inserted endoscopically into the frontal sinus. Rains indicates that his frontal sinus stent is self-retaining.

In practice, however, the Rains stent is very inflexible and upon its removal creates the circumferential injury that one is trying to prevent. Because it is relatively inflexible, it causes trauma to the delicate area that is to be stented, and as a result it theoretically results in a lower patency rate than softer stents. Additionally, it is also very large and very difficult to place.

Alternatively, a rolled piece of standard plastic sheeting (e.g., silastic sheeting) may be cut and rolled to be placed in the frontal sinus. See for example, Dubin et al., “Preservation of Natural Frontal Sinus Outflow in the Management of Frontal Sinus Osteomas,” Otolaryngology—Head and Neck Surgery, 2006, 134, 18-24; and Perloff et al., “Evidence of Bacterial Biofilms on Frontal Recess Stents in Patients with Chronic Rhinosinussitis,” American Journal of Rhinology, November-December 2004, vol. 18, no. 6, 377-380.

The silastic sheeting works well but can be difficult to place into the frontal sinus, particularly for those not adept at frontal sinus surgery whose patients would most benefit from stenting. It is not commercially available as a frontal stent, and is typically custom made from standard silastic sheeting. Furthermore, when it is placed, it is challenging to expand because the surface tension keeps the silastic in its original position (i.e., the memory of the silastic is less than the surface tension of the blood/saline in the nose). As a result, the silastic sheet that is currently used by some highly trained sinus surgeons is not commonplace, and even in skilled hands is not often used as it is difficult to place.

Thus, the field of frontal sinus surgery suffers from a lack of a self-expanding frontal sinus stent that may be easily placed in the frontal sinus drainage pathway. Such devices and methods are needed in order to maintain patency of the frontal sinus drainage pathway during the healing process following sinus surgery.

### SUMMARY

Certain embodiments of the present invention are directed to a self-expanding stent for the management of the frontal sinus postoperatively. The self-expanding stent may include a body comprising a thin flexible plastic sheet material. The body may have two side edges. The self-expanding stent may include a retention member in an upper portion of the body. At least one recoil mechanism may be included on the retention member, the recoil mechanism may have a memory and recoil action to cause the retention member to self-expand from a rolled or collapsed position. The self-expanding stent may include a stent member in a lower portion of the body. At least one recoil mechanism may be included on the stent member, the recoil mechanism may have a memory and recoil action to cause the stent member to self-expand from a rolled or collapsed position. A connector portion in a center region of the body may connect the retention member and the stent member. A slit may extend inward from each of the two side edges toward the center region of the body. The slits may separate the retention member and the stent member.

According to another aspect of the invention, the retention member further comprises an upper portion of the sheet material, wherein the retention member is flexible to be rolled and/or collapsed to pass through a narrow space of the nose, nasal cavity, and frontal sinus. According to another aspect of the invention, the stent member further comprises a lower portion of the sheet material, wherein the stent member is flexible to be rolled and/or collapsed to pass through a narrow space of the nose, nasal cavity, and frontal sinus.

In one embodiment, the self-expanding stent may comprise a rolled stent member formed by folding the side edges of at least the stent member inward toward the center region and overlapping the side edges. The at least one recoil member of the stent member acts to urge the rolled stent member to self-expand.

According to another aspect of the invention, a force exerted by the recoil mechanism is sufficient to overcome any surface tension, or frictional forces, or static forces of the overlapped side edges of the rolled stent member.

In another embodiment, the self-expanding stent may comprise a rolled retention member formed by folding at least the side edges of the retention member inward toward the center region and overlapping the side edges. The at least one recoil member of the retention member acts to urge the rolled retention member to self-expand.

According to another aspect of the invention, a force exerted by the recoil mechanism is sufficient to overcome any surface tension, or frictional forces, or static forces of the overlapped side edges of the rolled retention member.

In one embodiment, the recoil mechanisms comprises strips of material extending transversely across the body. For example, the strips of material may include a thicker piece of the same material as the material of the body, the thicker piece of the same material having a memory to return to a flat position. For example, the strip of material may include a piece of metal having a memory to return to a flat position.

In one embodiment, the recoil mechanism may be embedded in the material of the body. In another embodiment, the recoil mechanism is disposed on the body. In yet another embodiment, the recoil mechanism is attached to the body.

In another embodiment, the retention member includes anchors located proximate corners of the retention member between the side edges and the bottom edges.

In some embodiments, the body material comprises a medical grade, silicone elastomer material. One or more medications may be embedded within and/or coated on a surface of the self-expanding stent. The body material may include a thickness of between about 0.005-inch and about 0.03-inch.

According to another embodiment of the invention, the retention member further includes contoured bottom edges. The contoured bottom edges may include substantially straight edges that taper outward and upward at an angle from the center region of the body to the side edges of the body.

According to another aspect of the invention, the stent member includes at least two recoil mechanisms. For example, the at least two recoil mechanisms may include an upper recoil mechanism and a lower recoil mechanism.

According to another embodiment of the invention, a self-expanding frontal sinus stent is provided. The self-expanding frontal sinus stent includes a retention member for placement in the frontal sinus and a stent member for placement in the drainage pathway connecting the frontal sinus and the nasal cavity. The retention member may include a body having a top edge, two side edges, and two bottom edges, the body comprising a flexible material. A recoil mechanism may extend substantially from one side edge to the other side edge of the retention member. The retention member recoil mechanism includes a memory to self-expand when placed, thereby allowing the self-expanding frontal sinus stent to be self-retaining. The stent member may include a body having two side edges, the body comprising a flexible material. A rolled stent member may be formed by rolling the side edges of the body over one another so that the side edges overlap. The rolled stent member may have a substantially cylindrical shape. The rolled stent member may include a first opening located at a first end of the rolled stent member, a second opening located at a second end of the rolled stent member, one or more side walls extending between the first end and the second end of the rolled stent member, and a passageway defined by the one or more side walls, the passageway connecting the first opening and the second opening. A recoil mechanism may extend substantially from one side edge to the other side edge of the stent member. The stent member recoil mechanism includes a memory to self-expand when placed, thereby making the stent member self-expanding.

According to another aspect of the invention, at least a portion of the side edges overlap when the rolled stent member is unfurled to the maximum extent possible within a drainage pathway of the frontal sinus. According to another aspect of the invention, the rolled stent member is compliant, meaning the rolled stent member self-expands to fit within and fill a space, duct or pathway in which the rolled stent member is placed. According to another aspect of the invention, the recoil mechanisms allow the retention member and/or the stent member to be self-expanding from a rolled or collapsed position.

Another embodiment of the invention includes a method of stenting the drainage pathway of the frontal sinus. The method includes: providing a flat flexible sheet of medical grade plastic material; separating the sheet of material into a retention member and a stent member using slits in the flexible sheet of material, the slits extending from side edges of the sheet of material toward a center region; rolling at least the stent member by folding side edges of the stent portion toward a center region and overlapping the side edges; placing the self-expanding stent, wherein the retention member is located in the frontal sinus and the stent member is located in the frontal sinus drainage pathway; and self-expanding the rolled stent member to fill the drainage pathway between the frontal sinus and the nasal cavity.

According to another aspect of the invention, self-expanding is accomplished using a recoil mechanism having a memory to self-expanding from the rolled position to a flat position. The method may include forming the recoil mechanism from a thicker piece of the flat flexible sheet of medical grade plastic material having a memory, the memory exerting a force sufficient to overcome any surface tension, or frictional forces, or static forces of the overlapped side edges of the rolled stent member. Alternatively, the method may include forming the recoil mechanism from a piece of metal material having a memory, the memory exerting a force sufficient to overcome any surface tension, or frictional forces, or static forces of the overlapped side edges of the rolled stent member.

According to another aspect of the invention, the method may include endoscopically placing the self-expanding stent, wherein the retention member is placed in the frontal sinus and the stent member is placed in the frontal sinus drainage pathway.

Another embodiment of the invention is directed to an insertion tool for placing a self-expanding stent endoscopically into the frontal sinus. According to one embodiment, the insertion tool may include a tube-like housing having a proximal end and a distal end. A neck portion of the tube-like housing may be located between the proximal end and the distal end. A proximal opening may be located at the proximal end and a distal opening may be located at the distal end. A passageway may extend through the tube-like housing between the proximal opening and the distal opening. A cavity may be located at the distal end of the tube-like body and in communication with the passageway. In some embodiments, the cavity may include substantially the same diameter as the distal opening. A plunger may be slidably disposed within the passageway. An activator may be provided at a proximal end of the plunger, the activator being located external to the tube-like housing. A footplate may be located at a distal end of the plunger and within the cavity.

According to another aspect of the invention, the neck portion is flexible and malleable. According to another aspect of the invention, the neck portion is angled or curved.

According to another aspect of the invention, the cavity has a substantially constant diameter and is adapted to receipt a rolled self-expanding stent. The footplate may have a diameter slightly less than the cavity diameter to form a tight clearance between the footplate and a cavity sidewall. According to another embodiment, the footplate may include a raised edge around a periphery of the footplate.

According to another aspect of the invention, the tube-like housing comprises a substantially constant diameter. In another embodiment, the tube-like housing comprises a varying diameter, wherein the maximum diameter of the tube-like housing is at the distal end of the insertion tool in the area of the cavity.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention will become apparent from the following detailed description of the invention when considered in conjunction with the accompanying drawings. For the purpose of illustrating the invention, there is shown in the drawings embodiments that are presently preferred, it being understood, however, that the invention is not limited to the specific instrumentalities disclosed. In the drawings:

FIG. 1 is a front view of an exemplary self-expanding frontal stent placed in the frontal sinus;

FIG. 2 is a front view of an exemplary stent material in a flat position;

FIG. 3 is a front view of the exemplary stent material of FIG. 1 is a partially rolled position;

FIG. 4 is a front view of the exemplary stent material of FIG. 1 showing a retention member and a rolled stent member of the self-expanding stent;

FIG. 5 is an end view of the exemplary self-expanding stent of FIG. 3;

FIG. 6 is a front view of an exemplary self-expanding stent having a retention member with contoured bottom edges;

FIG. 7A is a side view of an exemplary insertion tool loaded with a self-expanding stent;

FIG. 7B is a side view of the exemplary insertion tool of FIG. 5A showing the self-expanding stent partially deployed;

FIGS. 8A and 8B show a side view and end view, respectively, of another exemplary insertion tool loaded with a self-expanding stent;

FIG. 9 is a detailed cross sectional view of the insertion tool and self-expanding stent of FIG. 8A showing the distal end of the insertion tool and a self-expanding stent loaded in the insertion tool;

FIG. 10 is the front view showing the insertion tool inserted through the nostril of a patient and into the frontal sinus and deployment of the self-expanding stent into the drainage pathway of the frontal sinus;

FIG. 11A is a cross-sectional view of the insertion tool inserted into the frontal sinus and the self-expanding stent not yet deployed;

FIG. 11B is a cross-sectional view of the insertion tool inserted into the frontal sinus and the self-expanding stent being deployed;

FIG. 11C is a cross-sectional view of the self-expanding stent deployed in the frontal sinus; and

FIGS. 12A-12C show details of exemplary embodiments of the distal end of the insertion tool.

### DETAILED DESCRIPTION OF ILLUSTRATIVE EMBODIMENTS

The present invention is directed to embodiments of self-expanding frontal sinus stent that overcomes the shortcomings of conventional stents and that may be used, for example, for the management of the frontal sinus postoperatively. In one embodiment, the self-expanding stent comprises a flexible plastic material having with one or more recoil mechanisms that have memory to self expand. One advantage of the self-expanding stent is that it is compliant, meaning that it may expand to the actual size of the space into which it is placed. In addition, embodiments of the present invention include an insertion tool with the appropriate angulation and malleability to facilitate ease of insertion of the self-expanding sinus stent in the frontal sinus. The use of a self-expanding frontal sinus stent and insertion tool for ease of insertion leads to less trauma for the patient and better surgical outcomes. Further, these features and improvements may lead to frontal sinus stenting becoming more commonplace in those cases where stenting is indicated.

The self-expanding frontal sinus stent serves at least two purposes: to help drainage from the frontal sinus; and to help maintain the opening or pathway connecting the frontal sinus cavity and the nasal cavity. The placement of the stent in this area physically occupies this space thus preventing the space from filling up with blood and, over time, helps overcome the body's desire to make the trauma area around the opening or pathway into a non-existing space (e.g., by scarring over and closing the opening). Without stenting, the trauma space may fill up with blood, which may then turn into scar tissue closing the opening.

The self-expanding stent in accordance with the various embodiments of the present invention also provides several benefits and advantages. For example, the stent is self-expanding. The self-expanding stent includes a recoil mechanism having a memory and recoil action to self-expand the rolled stent material once it has been placed. Also, the self-expanding stent is compliant in that it expands to fit the space or pathway in which it is placed. Accordingly, one or more standard size stents may be used in a variety of different size spaces, ducts, or pathways.

Other benefits and advantages include the self retaining feature of the self-expanding stent. The retention member expands in the frontal sinus and acts to hold the stent in place in the frontal sinus drainage pathway.

Preferably, the stent material is soft, pliable, and malleable so that the self-expanding stent may be atraumatically placed and removed. A flexible and pliable material allows the self-expanding stent to be easily inserted and placed in relatively tight or narrow spaces since the stent is capable of being rolled and collapsing on itself. Also, the flexible and pliable stent material may be easily removed, again because the stent is capable of collapsing on itself. Further, the self-expanding stent material preferably comprises medical grade and/or an inert material, allowing the stent to be inserted into the body and also allowing for longer term stenting.

In some embodiments, the stent material may comprise a flexible silicone elastomer sheeting. Preferably, the material is inert and/or medical grade and is suitable for use in humans and animals with no or minimal tissue reaction. SILASTIC® brand sheeting, for example, works well in the frontal sinus as a stent to limit scarring and recurrent obstruction. The material of the sinus stent may comprise medical grade SILASTIC® brand silicone elastomers, manufactured by Dow Corning Corporation.

For example, the stent material may comprise a pliable sheet of 1/100 inch thick SILASTIC® brand material. In some embodiments, the stent material may have a thickness between 0.005 inch and 0.03 inch.

Embodiments of the present invention provide an improvement by adding a recoil mechanism in the stent material, thus making the stent self-expanding when released from a rolled position. The recoil mechanism may include a thicker material (e.g., metal or plastic or other materials with more recoil/memory) embedded in or located on the stent material. This self-expanding feature of the sinus stent ensures that the stent opens fully in the drainage pathway (or outflow tract) of the frontal sinus and makes it significantly easier to place.

The self-expanding frontal sinus stent helps to maintain patency of the frontal sinus drainage pathway during the healing process thereby helping to solve the problem of restenosis of the frontal sinus drainage pathway following sinus surgery. This leads to improved success rates for both endonasal and external frontal sinus surgeries. A self-expanding frontal sinus stent may also reduce and/or prevent scarring associated with denuded bone in the frontal sinus.

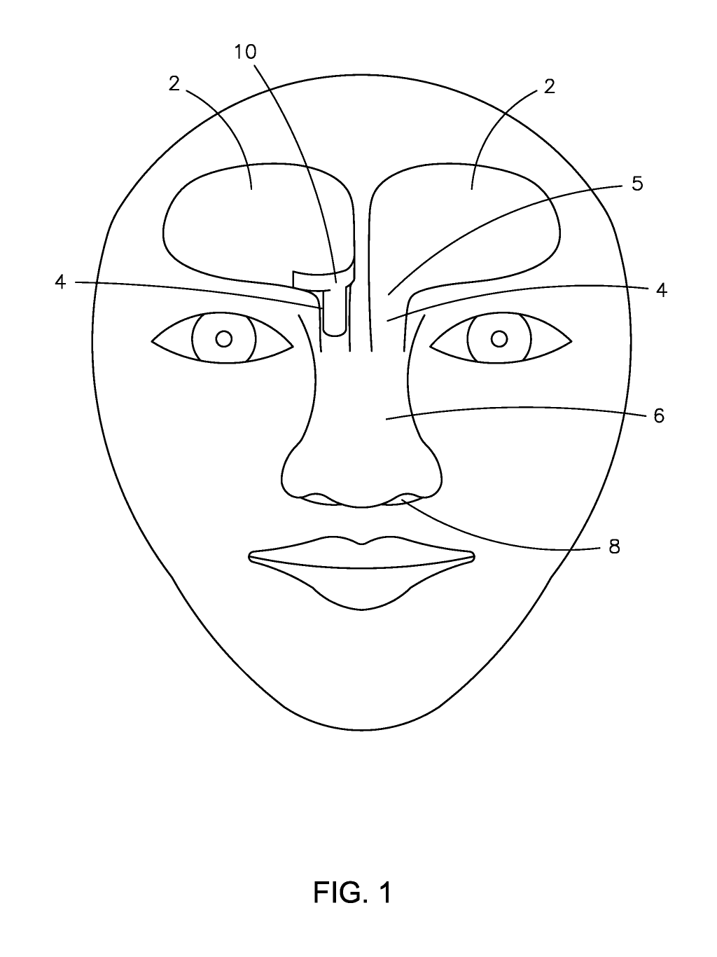

FIG. 1 shows an exemplary self-expanding frontal sinus stent 10 inserted in the frontal sinus 2 and the frontal sinus drainage pathway 4. The self-expanding frontal sinus stent 10 allows fluids to drain from the frontal sinus 2 into the nasal cavity 6. The self-expanding frontal sinus stent maintains patency of the pathway 4 connecting the frontal sinus 2 and the nasal cavity 6 during postoperative healing.

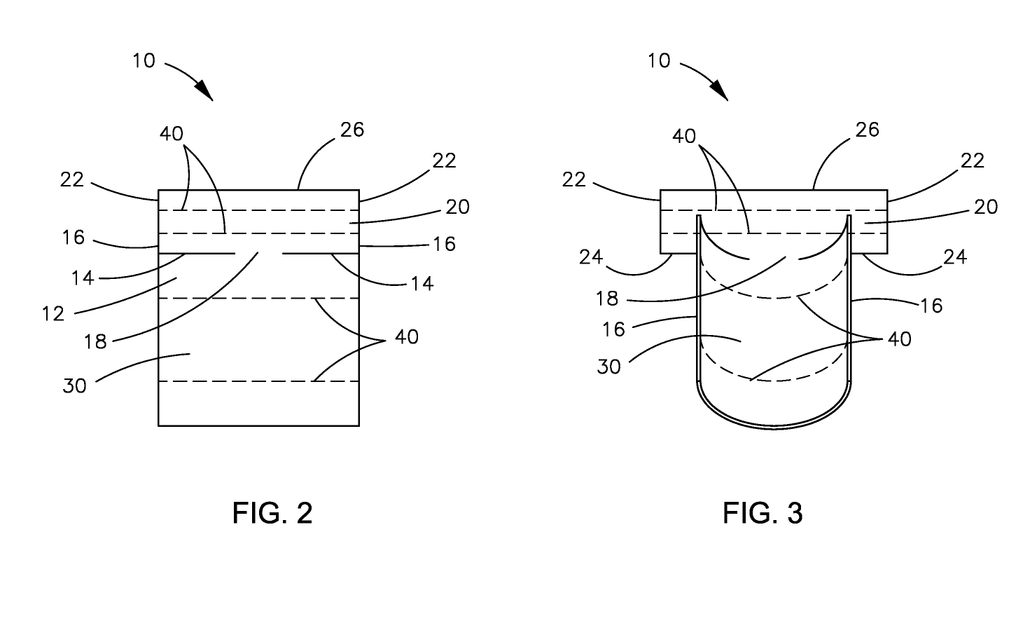

FIGS. 2-5 show an exemplary self-expanding frontal sinus stent 10. As shown, the stent may include a body 12, slits 14, a retention member 20, a stent member 30, and one or more recoil mechanisms 40. Prior to use, the stent 10 may include a flat sheet 12, as shown in FIG. 2. Slits 14 may be provided in the body 12 extending from a side edge 16 toward a center region of the body 12. As shown, the slits 14 do not extend all the way to the center, but rather stop proximate the center region of the body 12. The retention member 20 is location is an upper portion of the body 12 above the slits 14. The stent member 30 is located in a lower portion of the body 12 below the slits 14. As shown, a connector portion 18 connects the retention member 20 to the stent member 30.

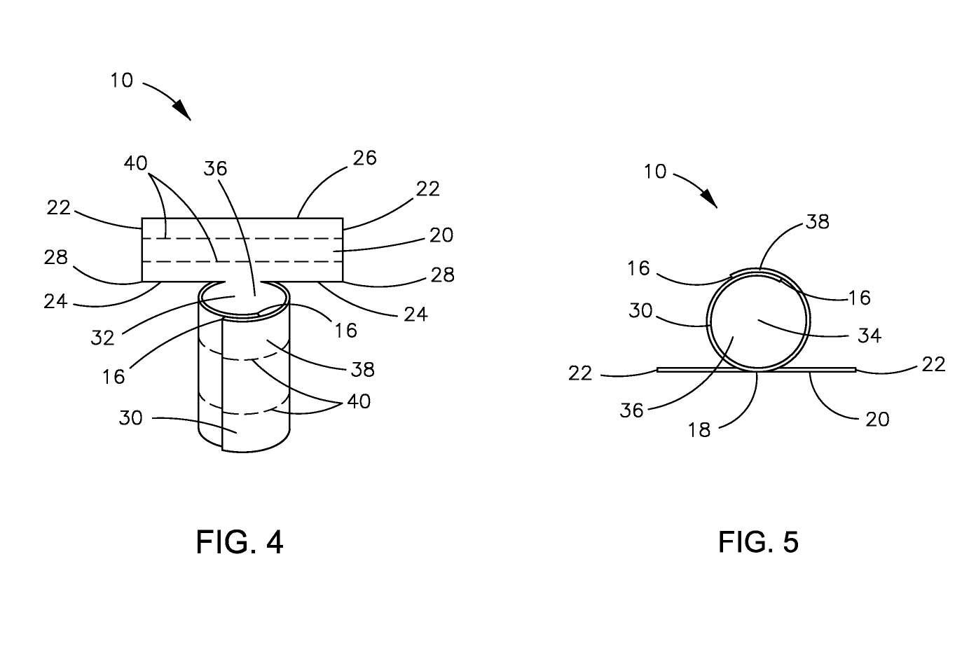

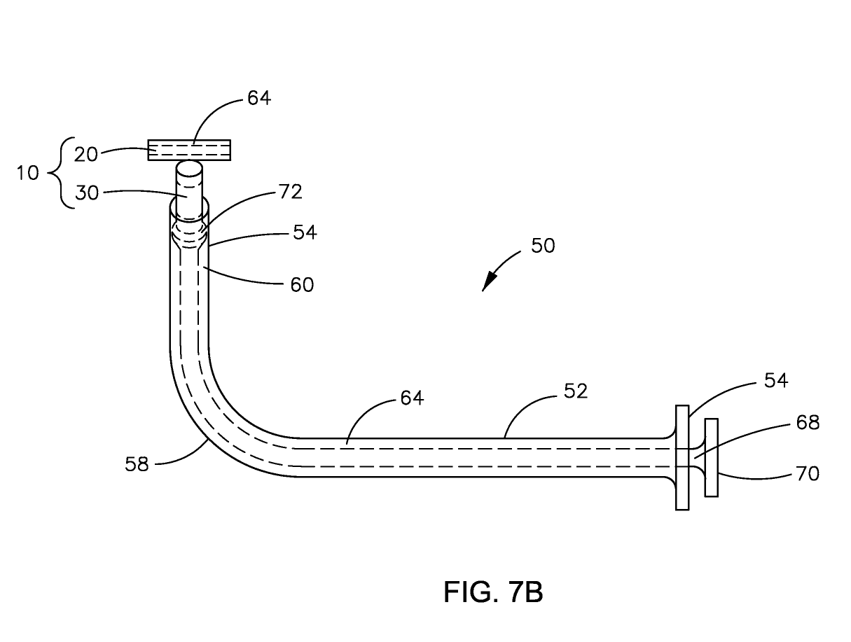

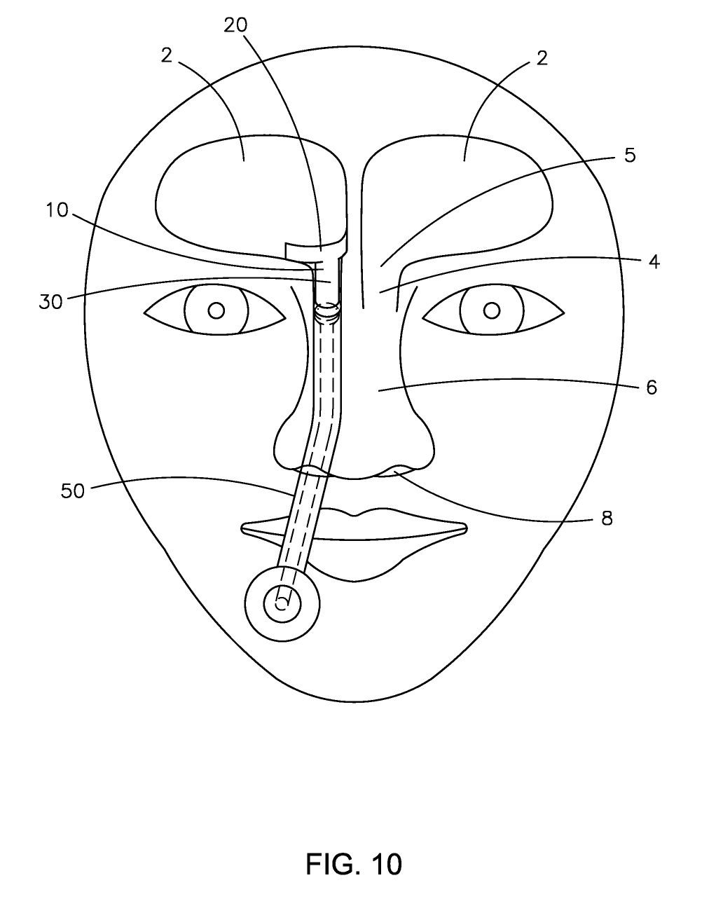

The retention member 20 allows the stent to be self-retaining. As shown in, FIGS. 2-5, the retention member 20 includes side edges 22, bottom edges 24 and a top edge 26. In some embodiments, the retention member 20 may be unrolled during placement (see e.g., FIGS. 3 and 4) and the material may be flexible enough to collapse and pass through narrow spaces (e.g., in the nose, nasal passageway, nasal cavity, frontal sinus pathway, frontal sinus, etc.) during placement. In other embodiments, the retention member may be rolled for placement, as shown for example in FIGS. 7A, 8A, 8B, 9, and 11A. When the self-expanding stent has been properly placed, the retention member 20 may be located in the frontal sinus 2, as shown, for example, in FIGS. 1, 10, 11B, and 11C. As shown, the bottom edges 24 may be positioned at the opening 5 of the frontal sinus drainage pathway 4. The retention member 20 may also include anchors 28. As shown, the anchors 28 may comprise the corners of the retention member 20 between the bottom edges 24 and the side edges 22. In other embodiments, the anchors may include hooks (not shown) to help hold the stent in place.

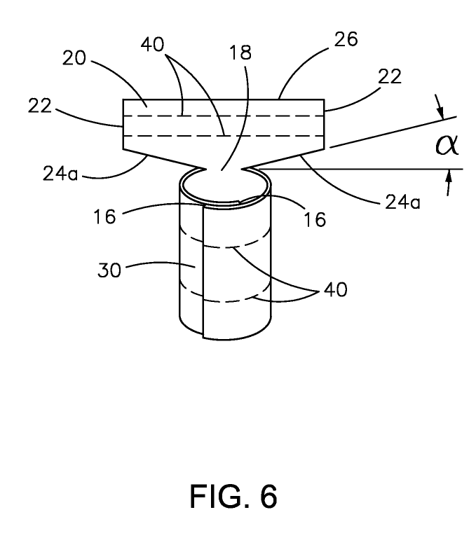

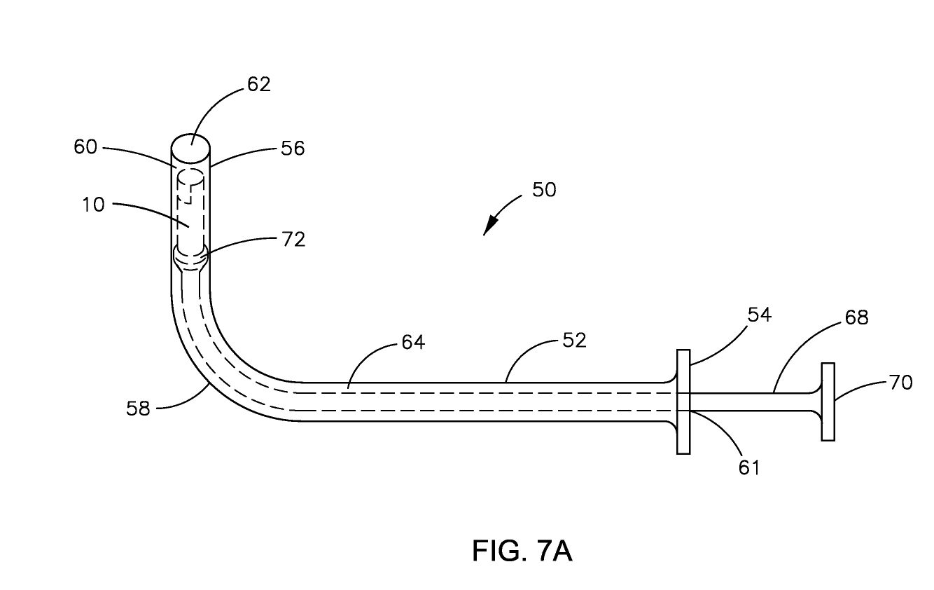

In some embodiment, the retention member 20 may include contoured bottom edges 24a, as shown in FIG. 6. As shown, the contoured edges 24a may be formed along the bottom edges of the retention member 20. The frontal sinus 2 is essentially shaped as an inverted funnel. This shape facilitates drainage of fluid from the frontal sinus 2. As such, in some embodiments it may be desirable to form the retention member 20 having contoured edges that substantially conform to the shape of the frontal sinus. As shown in FIG. 6, the contoured edges 24a may be formed at straight edges that extend outward and upward from the center region or connector portion 18 to the side edges 16 at an angle α. In another embodiment (not shown), the contoured edges may be formed as curved edges. The curved edges may be formed having a concave or a convex shape as viewed from the frontal sinus. In another embodiment (not shown), the contoured edges may comprise rounding the corners at the intersection of the slits and the side edges of the body. Forming the retention member with contoured bottom edges 24a may improve patient comfort.

As shown in FIGS. 3-5, the body 12 comprises a flexible material that may be rolled by folding in the side edges 16 toward the center region of the body 12. The side edges 16 may overlap one another and form a rolled stent member, as shown for example in FIGS. 4 and 5. As shown, the rolled stent member 30 may comprise a substantially cylindrical shape. The rolled stent member 30 has a first opening 32 located at a first end and a second opening 34 located at a second end of the rolled stent member 30. The first and second openings 32, 34 are connected by a passageway 36. The passageway 36 includes one or more side walls 38.

The material of the self-expanding stent 10 is preferably thin and flexible enough to collapse on itself allowing it to be placed. The stent body 12 may be tightly rolled upon itself to a couple of millimeters thick, or less, for placement in the drainage pathway 4 of the frontal sinus 2. The one or more recoil mechanisms 40 also allow the stent material to collapse for easy insertion and removal and also include a memory that allows the rolled stent to spring open once it is placed within the desired space, duct, or pathway. For example, the stent material 12 and recoil mechanism 40 may be rolled and collapsed for ease of placement and removal.

The recoil mechanism 40 causes the rolled material (e.g., rolled retention member 20 and/or rolled stent member 30) to attempt to spring open once released due to the memory of the material. The force exerted by the recoil mechanism 40 is preferably sufficient to overcome any surface tension, or frictional forces, or static forces of the rolled material. As such, the side edges 16 of the body 12 that were overlapped when the stent member 30 was rolled, slide over one another as the rolled stent member 30 unrolls and expands (i.e., as the diameter of the passageway 36 of the rolled stent member 30 increases). In other words, the rolled stent member 30 attempts to return to an unrolled or flat shape. The walls of the pathway 4 connecting the frontal sinus 2 and the nasal cavity 6, however, prevent the stent member from becoming completely unrolled or unfurled. As such, the overlap of the side edges 16 of the rolled stent member 30 may be such that the there is still some overlap of the side edges 16 when the stent member is unfurled to the maximum extent possible within the pathway 4. This overcomes the problem of prior art devices that failed to properly open or expand once the device was placed.

As shown the exemplary embodiments, the recoil mechanism 40 extends substantially transverse across the body. The recoil mechanism 40 may extent from one side edge to the other side edge. Other locations and orientations are contemplated, so long as the recoil mechanism has the effect of self-expanding the rolled stent member when released. In one embodiment, the recoil mechanism 40 may include the same plastic material as the stent body, but may include a thicker material with more recoil. In another embodiment, the recoil mechanism 40 may include a metal with recoil. The recoil mechanism 40 may be embedded into the stent material 12. The recoil mechanism 40 may be disposed on a surface of the stent material 12. In yet another embodiment, the recoil mechanism 40 may be attached to a surface of the stent material 12.

Once placed, the recoil mechanism 40 causes the retention member 20 to expand into the sinus cavity and also the stent member 30 to expand to fit the space, duct or pathway the self-expanding stent is placed within. Preferably, the stent opens to the maximum extent (e.g., maximum diameter) possible given the diameter of the pathway into which the stent is placed.

The diameter of the passageway 36 need not be constant. Preferably, more than one recoil mechanisms 40 are provided with the self expanding stent 10. For example, as shown in FIGS. 2-6, an upper and a lower recoil mechanism may be provided in the self-expanding stent 10. For example, as shown the self-expanding stent 10 may include two recoil mechanisms 40 on the retention member 20 and two recoil mechanisms 40 on the stent member 30. Preferably, the number and location of the recoil mechanisms 40 ensures that the stent 10 opens fully within the pathway 4 connecting the frontal sinus 2 and the nasal cavity 6.

The self-expanding frontal sinus stent may be used in the treatment of chronic frontal sinus disease. For example, stenting may be indicated following an osteoplastic flap procedure to keep the drainage pathway open and to prevent scarring of the residual anterior table mucosa to the posterior table. For example, stenting may be indicated following a primary endoscopic frontal sinus surgery for short term stenting (i.e., spacer). For example, stenting may be indicated following a primary endoscopic frontal sinus surgery for intermediate-long term stenting (e.g., 6 months to 1 year). For example, stenting may be indicated following a revision endoscopic sinus surgery for short or long term stenting. For example, stenting may be indicated following sphenoid sinus surgery (primary or revision). For example, stenting may be indicated following open frontal sinus surgery. For example, stenting may be indicated following trephination. For example, stenting may be indicated after balloon dilation. Use of a self-expanding stent following any of the preceding procedures/surgeries may help to prevent scarring and keep the drainage pathway open.

A self-expanding frontal sinus stent 10 may be left in place for a predetermined period of time. The period of time that the stent may be used will typically depend on the type of surgery, the indication for stenting, the anatomy of the patient, the condition of the patient's frontal sinus, etc. The desired time period is preferably determine to ensure patency of the frontal sinus (i.e., patent and functioning frontal ostia). For example, the predetermined period of time may comprise a short term or a long term. For example, short term stenting may include one or more weeks. For example, long term stenting may include multiple months. For example, stents may be left in place for about 9 months. For example, stents may be left in place for a time period of about 6 months to about 17 months. Stents placed after an osteoplastic flap may be left in place for about 12 months. For example, stents placed after an endoscopic procedure may be left in place for about 1 week to about 6 months.

In addition to the inclusion of a recoil mechanism with the stent material, the stent material may also be embedded with medications and/or medications may be applied to the surface of the stent material. For example, steroids, antibiotics, and the like may be embedded or applied to the surface of the stent material. Medications may be used to avoid or fight infection, ease pain and discomfort, aid the healing process, etc.

The self-expanding frontal sinus stent may be inserted in cases where stenting of the frontal sinus is indicated using an insertion tool 50, as depicted in FIGS. 7A-12C, or using other medical instruments.

As described in the exemplary embodiment disclosed above, the self-expanding frontal sinus stent 10 may be cut, rolled, placed, and unfurled to fill the drainage pathway 4 or frontal recess of the frontal sinus 2. The recoil mechanism 40 ensures that the self-expanding stent is fully unfurled. Preferably, the stent material is thin and flexible enough to collapse on itself allowing it to be placed and removed without trauma.

A medical instrument, for example, may be used to grab and remove the stent. For example, forceps, pincher, etc. may be used to grab, collapse, and remove the stent. In one embodiment, giraffe forceps (not show) may be used to place the self-expanding stent 10 in the frontal recess 4. For example, self-expanding stent 10 may be rolled and then grasped using giraffe forceps. The giraffe forceps may be used to place the self-expanding stent endoscopically into the drainage pathway 4 of the frontal sinus 2.

Embodiments of the insertion tool 50 may include a rigid and angled tool or a flexible tool. In either case, the insertion tool 50 may include a thin, tube-like instrument that may operate in a syringe-type manner to push the self-expanding stent 10 out of the insertion device once it is properly positioned within the frontal sinus 2.

Logistically, the frontal sinus cavity 2 and its opening is straight back and up from the nostrils 8. As such, performing sinus surgery and inserting the self-expanding stent endoscopically is akin to walking around while looking at the ceiling. Because of this, in one embodiment the insertion tool may be angled or curved. In another embodiment, the insertion tool may be flexible or malleable.

As shown in FIGS. 7A-12C, embodiments of the insertion tool 50 may include a tube-like housing 52 having a proximal end 54 and a distal end 56. The tube-like housing 52 may include a neck portion 58. In some embodiments, the neck portion 58 is angled, curved, and/or flexible/malleable.

As shown, the tube-like housing 52 may include a proximal opening 61 at the proximal end 54 of the device and a distal opening 62 at the distal end 56 of the device. A passageway 64 extends between and connecting the proximal opening 61 and the distal opening 62. A cavity 66 may be provided proximate the distal end 56 and in communication with the passageway 64 for receiving the rolled self-expanding stent 10.

As shown, a plunger 68 may be slidably disposed within the passageway 64 of the tube-like housing 52. The plunger 68 may include an activator 70 at the proximal end 54 of the device and a footplate 72 at the distal end 56. As shown, the activator 70 may be located external to the tube-like housing 52 and the footplate 72 may be slidably located within the cavity 66. The footplate 72 may include a surface for engaging the rolled self-expanding stent 10.

To load the rolled stent 10 into the insertion tool 50, the activator 70 of the plunger 68 may be withdrawn causing the footplate 72 to move to a lower position within the cavity 66. The rolled stent 10 may then be disposed within the cavity 66 and in contact with the footplate 72 through the distal opening 62. FIGS. 7A, 8A, 8B, 9 and 11A show the insertion tool 50 in a loaded condition.

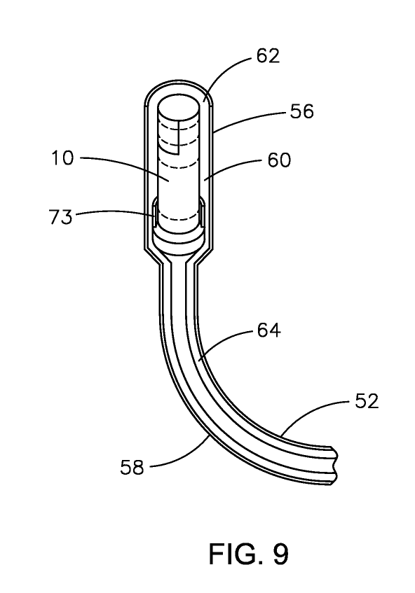

The rolled stent may or may not be secured to the footplate. As shown in the embodiment illustrated in FIG. 9, the footplate 72 may include a raised edge 73 around a periphery of the footplate the hold the self-expanding stent 10. In some embodiments, the rolled stent unrolls once it is placed within the cavity until it contacts sidewalls of the cavity 66. In this manner, the pressure the self-expanding stent 10 exerts on the sidewalls of the cavity 66 acts to hold the rolled self-expanding stent 10 in place within the cavity 66.

To activate the insertion tool 50 and place the self-expanding stent 10 in the desired location, the actuator 70 may be depressed. The inward movement of the actuator 70 causes the plunger 68 to move within passageway 64 and the footplate 72 to slide within cavity 66 toward the distal opening 62. Movement of the footplate 72 causes the rolled self-expanding stent 10 to exit the insertion tool 50 through distal opening 62.

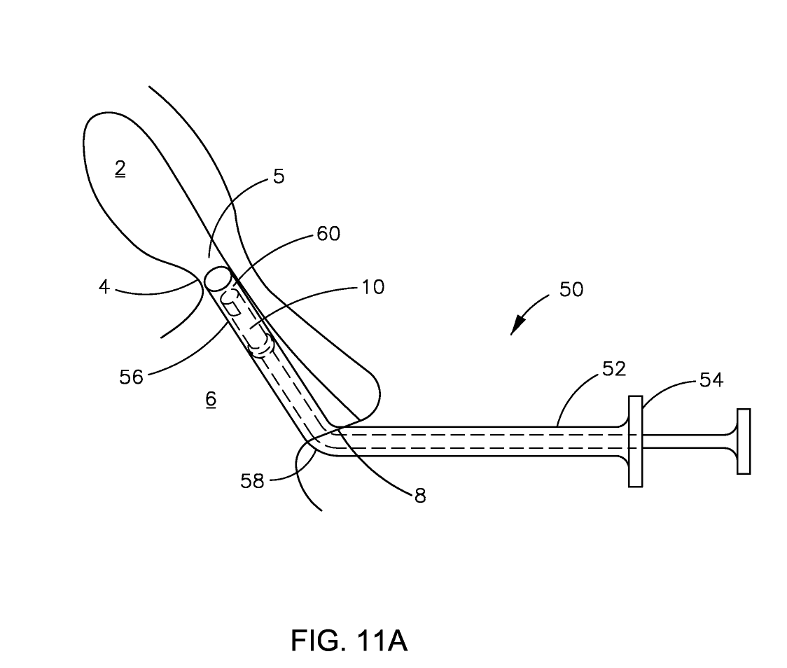

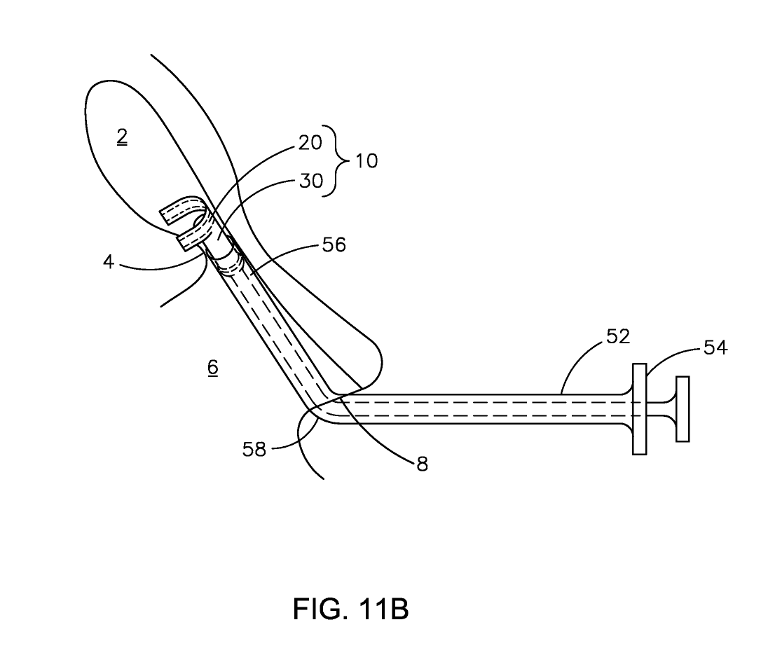

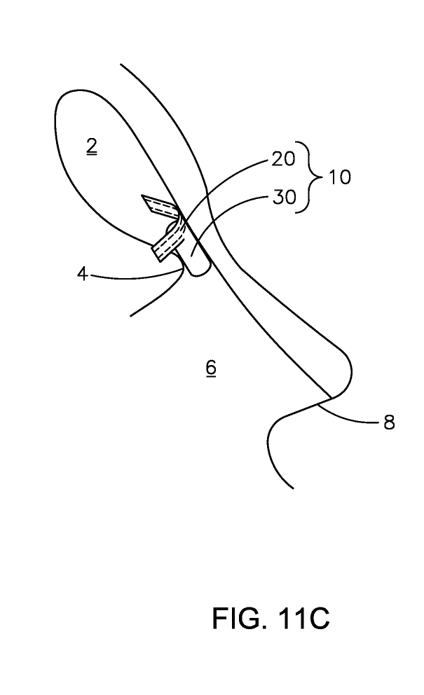

When the retention member 20 clears the rim of the distal opening 62, the recoil mechanism(s) 40 on the retention member 20 cause the rolled retention member 20 to open up/self-expand. The retention member 20 will open to fill the space, duct or pathway it is located in. In one embodiment, the retention member 20 may be located in the frontal sinus 2. FIGS. 7B and 11B show the insertion tool 50 placing the self-expanding stent 10 with the retention member 20 open/expanded.

Activation of the actuator 70 may continue and when the stent member 30 clears the rim of the distal opening 62, the recoil mechanism(s) 40 on the stent member 30 cause the rolled stent member 30 to open up/self-expand. The stent member 30 will self-expand to fill the space, duct or pathway it is location in. In one embodiment, the stent member 30 may be located in the pathway 4 connecting the frontal sinus 2 and the nasal cavity 6. FIGS. 1, 10 and 11C show the self-expanding stent 10 properly place and the retention member 20 and stent member 30 open/expanded.

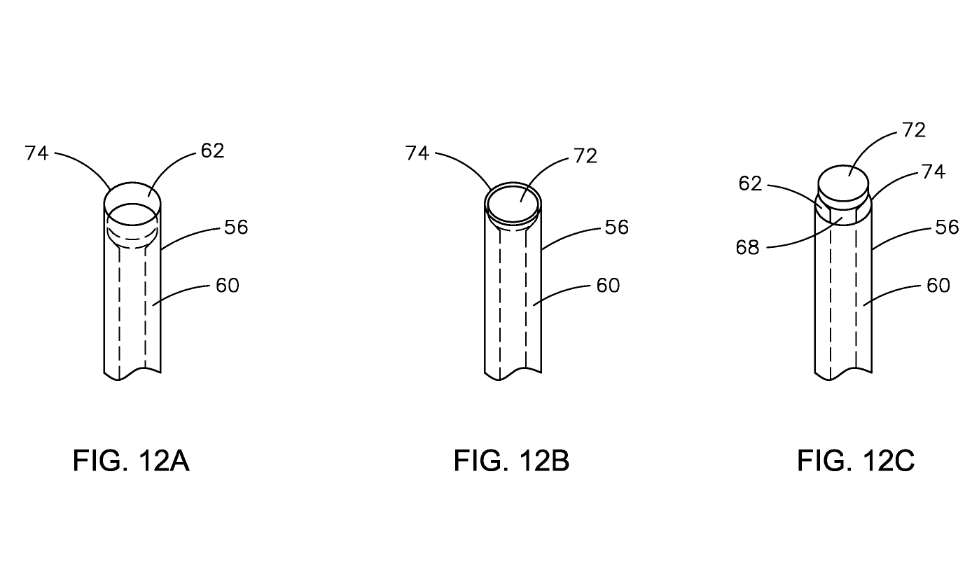

As shown in FIGS. 12A-12C, the footplate 72 may stop below the rim 74 of the distal opening 62 (FIG. 12A), even with the rim 74 of the distal opening 62 (FIG. 12B), or may extend beyond the rim 74 of the distal opening 62 (FIG. 12C). For example, once the retention member 20 is pushed out of the cavity 66 and unrolls or expands, it may engage the walls of the frontal sinus 2 and the insertion tool 50 may be pulled down to complete the deployment of the rolled self-expanding stent 10 from the insertion tool 50. Extending the footplate 72 beyond the rim 74 of the distal opening 62 may be helpful in ensuring that the rolled self-expanding stent 10 is successfully pushed out of the insertion tool 50.

Preferably, the footplate 72 forms a snug fit within the sidewalls of the cavity 66 (i.e., a tight clearance between the footplate and a sidewall of the cavity). This helps ensure that the footplate 72 contacts the rolled self-expanding stent 10 and pushes the rolled self-expanding stent 10 out through the distal opening 62 when the plunger 68 is activated.

Preferably, the edges 74 of the distal opening 62 are rounded to reduce irritating the nostrils, nasal passageway, nasal cavity, and/or frontal sinus pathway during placement of the self-expanding stent 10.

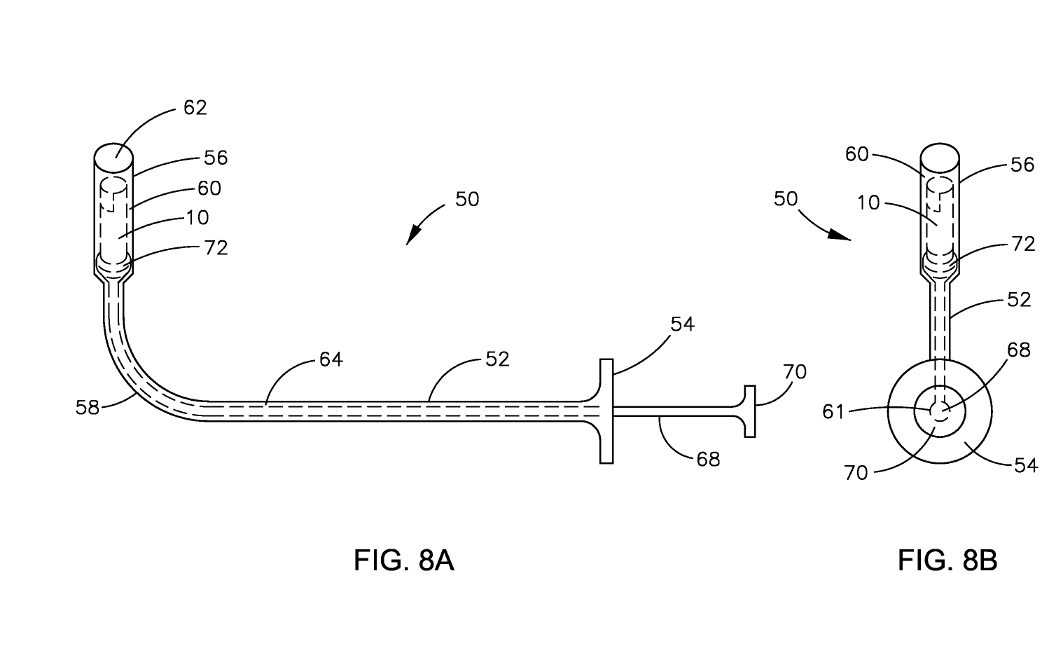

As shown in FIGS. 7A and 7B, the tube-like housing may include a substantially constant cross-section area or diameter. In some embodiments, the outside diameter of the tube-like housing may be minimized to the facilitate placement of the self expanding stent without causing any trauma. For example, as shown in FIGS. 8A and 8B, the portion of the tube-like housing up to the cavity at the distal end may include a smaller cross-sectional area or diameter that the cavity portion.

For example, the insertion tool 50 may be used to place a self-expanding stent in a space, duct, pathway, etc. having a diameter of approximately 4 mm-20 mm. In one embodiment, the length of insertion tool when straight may be about 10 cm, an outer diameter of the distal end of the insertion tool may be about 8 mm, and an inner diameter of the distal end of the insertion tool may be about 6 mm. The insertion tool may come in various sizes to be compatible for use with different patients.

The insertion tool 50 may be used to insert the self-expanding frontal sinus stent 10 endoscopically. The insertion tool 50 may be inserted though one of the nostrils 8 of the patient. A nasal decongestant or numbing medicine may be applied topically in the patient's nose prior to insertion of the tool. The insertion tool 50 may be fed or manipulated into the patient's nasal passage and nasal cavity 6 until into distal end 56 of the insertion tool 50 is positioned within or above the drainage pathway of the front sinus 4.

Once in position, the plunger 68 of the insertion tool 50 may be activated to deploy the self-expanding stent 10 in the frontal sinus drainage pathway 4. As the plunger 68 is depressed, the self-expanding stent 10 is pushed out of the opening in the distal end 56 of the insertion tool 50. The retention member 20 exits the tip opening 62 and expands into the frontal sinus cavity 2. The retention member 20 holds the stent 10 in position. The plunger 68 continues to be depressed and the stent member 30 of the stent 10 passes through the tip opening 62. The stent member 30 self-expands as it clears the insertion tool 50.

The insertion tool 50 may be used in conjunction with a nasal endoscope and/or other nasal instruments or tools (not shown). For example, the endoscope may provide for suction to remove blood and other debris from the wound site and sinus drainage pathway during or just prior to insertion of the self-expanding frontal sinus stent. For example, the endoscope may provide a camera and lighting for improved visualization during the insertion process. In addition, the insertion tool may be used with an image guidance navigation system to guide the surgeon during placement of the self-expanding stent.

Although described with reference to use following frontal sinus surgery; embodiments of the present invention may also find use in applications following sphenoid sinus surgery.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit of the invention.

What is claimed:

1. A self-expanding stent comprising:
a body comprising a thin flexible plastic sheet material, the body having two side edges;
a retention member in an upper portion of the body;
at least one recoil mechanism on the retention member, the recoil mechanism having a memory and recoil action to cause the retention member to self-expand from a rolled or collapsed position;
a stent member in a lower portion of the body;
at least one recoil mechanism on the stent member, the recoil mechanism having a memory and recoil action to cause the stent member to self-expand from a rolled or collapsed position;
a connector portion in a center region of the body, the connector portion connecting the retention member and the stent member; and
a slit extending inward from each of the two side edges toward the center region of the body, the slits separating the retention member and the stent member.

2. The self-expanding stent of claim 1, further comprising a rolled stent member formed by folding the side edges of at least the stent member inward toward the center region and overlapping the side edges, the at least one recoil member of the stent member urging the rolled stent member to self-expand.

3. The self-expanding stent of claim 2, wherein a force exerted by the recoil mechanism is sufficient to overcome any surface tension, or frictional forces, or static forces of the overlapped side edges of the rolled stent member.

4. The self-expanding stent of claim 2, further comprising a rolled retention member formed by folding at least the side edges of the retention member inward toward the center region and overlapping the side edges, the at least one recoil member of the retention member urging the rolled retention member to self-expand.

5. The self-expanding stent of claim 4, wherein a force exerted by the recoil mechanism is sufficient to overcome any surface tension, or frictional forces, or static forces of the overlapped side edges of the rolled retention member.

6. The self-expanding stent of claim 1, wherein the recoil mechanisms further comprise strips of material extending transversely across the body.

7. The self-expanding stent of claim 6, wherein strips of material comprising the recoil mechanisms further comprise a thicker piece of the same material as the material of the body, the thicker piece of the same material having a memory to return to a flat position.

8. The self-expanding stent of claim 6, wherein the strip of material comprising the recoil mechanism further comprises a piece of metal having a memory to return to a flat position.

9. The self-expanding stent of claim 1, wherein the recoil mechanism is embedded in the material of the body.

10. The self-expanding stent of claim 1, wherein the recoil mechanism is disposed on the body.

11. The self-expanding stent of claim 1, wherein the recoil mechanism is attached to the body.

12. The self-expanding stent of claim 1, the retention member further comprises anchors located proximate corners of the retention member between the side edges and the bottom edges.

13. The self-expanding stent of claim 1, wherein the body material comprises a medical grade, silicone elastomer material.

14. The self-expanding stent of claim 13, further comprising one or more medications embedded within and/or coated on a surface of the self-expanding stent.

15. The self-expanding stent of claim 1, wherein the body material has a thickness of between about 0.005-inch and about 0.03-inch.

16. The self-expanding stent of claim 1, wherein the retention member further comprises contoured bottom edges.

17. The self-expanding stent of claim 16, wherein the contoured bottom edges further comprise substantially straight edges that taper outward and upward at an angle from the center region of the body to the side edges of the body.

18. The self-expanding stent of claim 1, wherein the retention member further comprises an upper portion of the sheet material, wherein the retention member is flexible to be rolled and/or collapsed to pass through a narrow space of the nose, nasal cavity, and frontal sinus.

19. The self-expanding stent of claim 1, wherein the stent member further comprises a lower portion of the sheet material, wherein the stent member is flexible to be rolled and/or collapsed to pass through a narrow space of the nose, nasal cavity, and frontal sinus.

20. The self-expanding stent of claim 1, wherein the stent member further comprises at least two recoil mechanisms, the at lease two recoil mechanisms comprising an upper recoil mechanism and a lower recoil mechanism.

21. A self-expanding frontal sinus stent comprising:
a retention member for placement in the frontal sinus, the retention member comprising:
a body having a top edge, two side edges, and two bottom edges, the body comprising a flexible material;
a recoil mechanism extending substantially from one side edge to the other side edge of the retention member;
wherein the retention member recoil mechanism has a memory to self-expand when placed, thereby allowing the self-expanding frontal sinus stent to be self-retaining;
a stent member for placement in the drainage pathway connecting the frontal sinus and the nasal cavity, the stent member comprising:
a body having two side edges, the body comprising a flexible material;
a rolled stent member formed by rolling the side edges of the body over one another so that the side edges overlap, the rolled stent member comprising a substantially cylindrical shape;
a first opening located at a first end of the rolled stent member;
a second opening located at a second end of the rolled stent member;
one or more side walls extending between the first end and the second end of the rolled stent member;
a passageway defined by the one or more side walls, the passageway connecting the first opening and the second opening;
a recoil mechanism extending substantially from one side edge to the other side edge of the stent member;
wherein the stent member recoil mechanism has a memory to self-expand when placed, thereby making the stent member self-expanding; and
a connector portion connecting the retention member and the stent member.

22. The self-expanding frontal sinus stent of claim 21, wherein at least a portion of the side edges overlap when the rolled stent member is unfurled to the maximum extent possible within a drainage pathway of the frontal sinus.

23. The self-expanding frontal sinus stent of claim 21, wherein the rolled stent member is compliant, meaning the rolled stent member self-expands to fit within and fill a space, duct or pathway in which the rolled stent member is placed.

24. The self-expanding frontal sinus stent of claim 21, wherein the recoil mechanisms allow the retention member and the stent member to be self-expanding from a rolled or collapsed position.

25. A method of stenting the drainage pathway of the frontal sinus, the method comprising:
providing a flat flexible sheet of medical grade plastic material;
separating the sheet of material into a retention member and a stent member using slits in the flexible sheet of material, the slits extending from side edges of the sheet of material toward a center region;
rolling at least the stent member by folding side edges of the stent portion toward a center region and overlapping the side edges;
placing the self-expanding stent, wherein the retention member is located in the frontal sinus and the stent member is located in the frontal sinus drainage pathway; and
self-expanding the rolled stent member to fill the drainage pathway between the frontal sinus and the nasal cavity.

26. The method of claim 25, wherein the self-expanding is accomplished using a recoil mechanism having a memory to self-expanding from the rolled position to a flat position.

27. The method of claim 25, further comprising forming the recoil mechanism from a thicker piece of the flat flexible sheet of medical grade plastic material having a memory, the memory exerting a force sufficient to overcome any surface tension, or frictional forces, or static forces of the overlapped side edges of the rolled stent member.

28. The method of claim 25, further comprising forming the recoil mechanism from a piece of metal material having a memory, the memory exerting a force sufficient to overcome any surface tension, or frictional forces, or static forces of the overlapped side edges of the rolled stent member.

29. The method of claim 25, further comprising endoscopically placing the self-expanding stent, wherein the retention member is placed in the frontal sinus and the stent member is placed in the frontal sinus drainage pathway.

30. An insertion tool for placing a self-expanding stent endoscopically into the frontal sinus, the insertion tool comprising:
a tube-like housing having a proximal end and a distal end;
a neck portion of the tube-like housing located between the proximal end and the distal end;
a proximal opening located at the proximal end;
a distal opening located at the distal end;
a passageway that extends through the tube-like housing between the proximal opening and the distal opening;
a cavity located at the distal end of the tube-like body and in communication with the passageway, the cavity having substantially the same diameter as the distal opening;
a plunger slidably disposed within the passageway;
an activator located at a proximal end of the plunger, the activator being located external to the tube-like housing; and
a footplate located at a distal end of the plunger and within the cavity.

31. The insertion tool of claim 30, wherein the neck portion is flexible and malleable.

32. The insertion tool of claim 30, wherein the neck portion is angled or curved.

33. The insertion tool of claim 30, wherein the cavity has a substantially constant diameter and is adapted to receive a rolled self-expanding stent, wherein the footplate has a diameter slightly less than the cavity diameter to form a tight clearance between the footplate and a cavity sidewall.

34. The insertion tool of claim 30, wherein the footplate further comprises a raised edge around a periphery of the footplate.

35. The insertion tool of claim 30, wherein the tube-like housing comprises a substantially constant diameter.

36. The insertion tool of claim 30, wherein the tube-like housing comprises a varying diameter, wherein the maximum diameter of the tube-like housing is at the distal end of the insertion tool in the area of the cavity.
